(19) United States
(12) Patent Application Publication
(10) Pub. No.: US 2005/0080100 A1
(43) Pub. Date: Apr. 14, 2005

(54) Pyridylamino compounds and methods of use thereof

(75) Inventor: John Adams Lowe III, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/926,493

(22) Filed: Aug. 26, 2004

Related U.S. Application Data
(60) Provisional application No. 60/510,016, filed on Oct. 9, 2003.

(51) Int. Cl. A61K 31/513; C07D 213/62

(52) U.S. Cl. 514/269; 514/349; 544/317; 546/300

(57) ABSTRACT

This invention relates to a series of pyridylamino compounds of the formula I wherein Z₁, Z₂, W, X, Y and R₁ are defined as in the specification, that exhibit activity as glycine transport inhibitors, their pharmaceutically acceptable salts, pharmaceutical compositions containing them, and their use for the enhancement of cognition and the treatment of the positive and negative symptoms of schizophrenia and other psychoses in mammals, including humans.

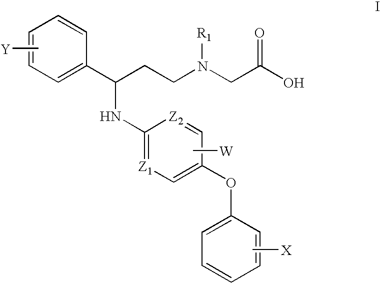

### BACKGROUND OF THE INVENTION

The present invention relates to pyridylamino compounds containing a pendant amino acid side chain and to pharmaceutical compositions containing them and to their use in the treatment of central nervous system disorders, cognitive disorders, schizophrenia, dementia and other disorders in mammals, including humans. These compounds exhibit activity as inhibitors of the glycine type-1 transporter.

Pharmacological treatment for schizophrenia has traditionally involved blockade of the dopamine system, which is thought to be responsible for its positive symptoms. Such treatment, however, ignores the negative and cognitive aspects of the disease. Another neurotransmitter system believed to play a role in schizophrenia is the glutamate system, the major excitatory transmitter system in the brain. This hypothesis is based on the observation that blockade of the glutamate system by compounds such as PCP (“angel dust”) can replicate many of the symptoms of schizophrenia, including its positive, negative, and cognitive aspects. If schizophrenia involves a deficit of glutamatergic transmission, augmentation of the glutamate system, and specifically the NMDA receptor, may be beneficial. While glutamate is the principal agonist at NMDA receptors, glycine is required as a co-agonist for the receptor's response to glutamate. Enhancing the effect of glycine would augment NMDA neurotransmission, and provide potential benefit in the treatment of schizophrenia.

A specific mechanism for augmenting the glycinergic effect was disclosed recently by Bergeron, et al. (Proc. Natl. Acad. Sci. USA, 95, 15730, (1998)), who showed that an inhibitor of the glycine type-1 transporter (GlyT1) responsible for removing glycine from the synapse at the NMDA receptor may enhance NMDA receptor function. This inhibitor is also described in WO 97/45115. The Bergeron et al. article and WO 97/45115 show that a GlyT1 inhibitor may be used to treat schizophrenia through its augmentation of glutamatergic neurotransmission.

### SUMMARY OF THE INVENTION

The present invention relates to a series of compounds of the formula I 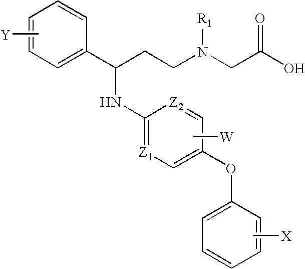
wherein Z₁ and Z₂ are independently carbon or nitrogen, provided that Z₂ is not carbon when Z₁ is carbon; and
W, X and Y are each, independently, hydrogen, (C₁-C₆) alkyl optionally substituted with from one to seven fluorine atoms, (C₁-C₆)alkoxy optionally substituted with from one to seven fluorine atoms, with the proviso that the number of fluorine substituents on each of the (C₁-C₆) alkyl and (C₁-C₆) alkoxy groups does not exceed the number of positions in each of the (C₁-C₆) alkyl and (C₁-C₆) alkoxy groups that are available for substitution; carboxy; carbo-(C₁-C₆)alkoxy; carboxamido; (C₁-C₆)alkyl-thio; alkylsulfoxyl; alkysulfonyl; halo; nitro; cyano; amino; (C₁-C₆) alkylamino and di[(C₁-C₆) alkyl]amino; and
R₁ is hydrogen or C₁-C₆alkyl;
or the pharmaceutically acceptable salts of such compounds.

In an exemplary embodiment of this invention, X is para-trifluoromethyl, para-methyl or para-chloro. In another exemplary embodiment, Z₁ is nitrogen and Z₂ is carbon. In another exemplary embodiment, W, X and Y are each, independently, hydrogen, (C₁-C₆) alkyl optionally substituted with from one to seven fluorine atoms, (C₁-C₆)alkoxy optionally substituted with from one to seven fluorine atoms, with the proviso that the number of fluorine substituents on each of the (C₁-C₆) alkyl and (C₁-C₆) alkoxy groups does not exceed the number of positions in each of the (C₁-C₆) alkyl and (C₁-C₆) alkoxy groups that are available for substitution; alkylsulfonyl; halo; (C₁-C₆) alkylamino and di[(C₁-C₆) alkyl]amino. For example, In another exemplary embodiment, R₁ is methyl.

Exemplary compounds of the invention include:
{Methyl-[3-phenyl-3-(5-p-tolyloxy-pyridin-2-ylamino)-propyl]-amino}-acetic acid;
({3-[5-(4-Methoxy-phenoxy)-pyridin-2-ylamino]-3-phenyl-propyl}-methyl-amino)-acetic acid;
({3-(4-Fluoro-phenyl)-3-[5-(4-methoxy-phenoxy)-pyridin-2-ylamino]-propyl}-methyl-amino)-acetic acid;
{[3-(4-Fluoro-phenyl)-3-(5-p-tolyloxy-pyridin-2-ylamino)-propyl]-methyl-amino}-acetic acid;
{[3-(4-Methoxy-phenyl)-3-(5-p-tolyloxy-pyridin-2-ylamino)-propyl]-methyl-amino}-acetic acid;
({3-[5-(4-Chloro-phenoxy)-pyridin-2-ylamino]-3-phenyl-propyl}-methyl-amino)-acetic acid;
{[3-[5-(4-Chloro-phenoxy)-pyridin-2-ylamino]-3-(4-fluoro-phenyl)-propyl]-methyl-amino}-acetic acid;
({3-[5-(3-Methoxy-phenoxy)-pyridin-2-ylamino]-3-phenyl-propyl}-methyl-amino)-acetic acid;
({3-(4-Fluoro-phenyl)-3-[5-(3-methoxy-phenoxy)-pyridin-2-ylamino]-propyl}-methyl-amino)-acetic acid;
({3-[5-(4-Fluoro-phenoxy)-pyridin-2-ylamino]-3-phenyl-propyl}methyl-amino)-acetic acid;
{[3-[5-(4-Fluoro-phenoxy)-pyridin-2-ylamino]-3-(4-fluoro-phenyl)-propyl]-methyl-amino}-acetic acid;
({3-[5-(3-Fluoro-phenoxy)-pyridin-2-ylamino]-3-phenyl-propyl}-methyl-amino)-acetic acid;
{[3-[5-(3-Fluoro-phenoxy)-pyridin-2-ylamino]-3-(4-fluoro-phenyl)-propyl]-methyl-amino}-acetic acid;
({3-[5-(4-Methoxy-phenoxy)-6-methyl-pyridin-2-ylamino]-3-phenyl-propyl}-methyl-amino)-acetic acid;
({3-(4-Fluoro-phenyl)-3-[5-(4-methoxy-phenoxy)-6-methyl-pyridin-2-ylamino]-propyl}-methyl-amino)-acetic acid;
({3-[5-(4-Methoxy-phenoxy)-4-methyl-pyridin-2-ylamino]-3-phenyl-propyl}-methyl-amino)-acetic acid; and
({3-(4-Fluoro-phenyl)-3-[5-(4-methoxy-phenoxy)-4-methyl-pyridin-2-ylamino]-propyl}-methyl-amino)-acetic acid.

Other embodiments of the invention include:
{Methyl-[3-phenyl-3-(5-p-tolyloxy-pyrimidin-2-ylamino)-propyl]-amino}-acetic acid;
({3-[5-(4-Methoxy-phenoxy)-pyrimidin-2-ylamino]-3-phenyl-propyl}-methyl-amino)-acetic acid;
({3-(4-Fluoro-phenyl)-3-[5-(4-methoxy-phenoxy)-pyrimidin-2-ylamino]-propyl}-methyl-amino)-acetic acid;
{[3-(4-Fluoro-phenyl)-3-(5-p-tolyloxy-pyrimidin-2-ylamino)-propyl]-methyl-amino}-acetic acid;
{[3-(4-Methoxy-phenyl)-3-(5-p-tolyloxy-pyrimidin-2-ylamino)-propyl]-methyl-amino}-acetic acid; and
({3-[5-(4-Chloro-phenoxy)-pyrimidin-2-ylamino]-3-phenyl-propyl}-methyl-amino)-acetic acid.

This invention also relates to:
a) a method of treating a disorder or condition selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders such as acute mania or depression associated with bipolar disorder and mood disorders associated with schizophrenia, behavioral manifestations of mental retardation, conduct disorder and autistic disorder; movement disorders such as Tourette's syndrome, akinetic-rigid syndrome, movement disorders associated with Parkinson's disease, tardive dyskinesia and other drug induced and neurodegeneration based dyskinesias; neurodegenerative diseases such as multiple sclerosis; attention deficit hyperactivity disorder; cognitive disorders such as dementias (including age related dementia, and senile dementia of the Alzheimer's type) and memory disorders in a mammal, including a human, comprising administering to a mammal in need of such treatment an amount of a compound of the formula I, or a pharmaceutically acceptable salt thereof, that is effective in treating such condition or disorder;
b) a pharmaceutical composition for treating a disorder or condition selected from the disorders or conditions described in the previous paragraph, comprising a compound of the formula I, or a pharmaceutically acceptable salt thereof, in an amount that is effective for treating such disorder or condition;
c) a method for treating a disorder or condition selected from the disorders or conditions described in the previous paragraph, comprising administering to a mammal in need of such treatment a glycine transport-inhibiting amount of a compound of the formula I, or a pharmaceutically acceptable salt thereof; and
d) a pharmaceutical composition for treating a disorder or condition selected from the disorders or conditions described in the previous paragraph, comprising a compound of the formula I, or a pharmaceutically acceptable salt thereof, in a glycine transport-inhibiting amount.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso-butyl, sec-butyl, tert-butyl, pentyl, hexyl, heptyl, 3-ethylbutyl, cyclopropyl, cyclobutyl, cyclopentyl, cyclohexyl, cycloheptyl, norbornyl, and the like.

The term “halo”, as used herein, means chloro, fluoro, iodo or bromo.

The term “alkoxy”, as used herein, means “alkyl-O—”, wherein “alkyl” is defined as above.

The term “treating”, as used herein, refers to reversing, alleviating, inhibiting the progress of, or preventing the disorder or condition to which such term applies, or one or more symptoms of such condition or disorder. The term “treatment”, as used herein, refers to the act of treating, as “treating” is defined immediately above.

The compounds of formula I may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, 18O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritium and ¹⁴C isotopes are particularly preferred for their ease of preparation and detectability. Further, substitution with heavier isotopes such as deuterium can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Scheme and/or in the Examples and Preparations below, by substituting a readily available isotopically labelled reagent for a non-isotopically labelled reagent.

### DETAILED DESCRIPTION OF THE INVENTION

The compounds of the formula I of this invention may be prepared as described in the following reaction schemes. Unless otherwise indicated, in the reaction schemes and discussion that follow, W, X and Y are defined as above.

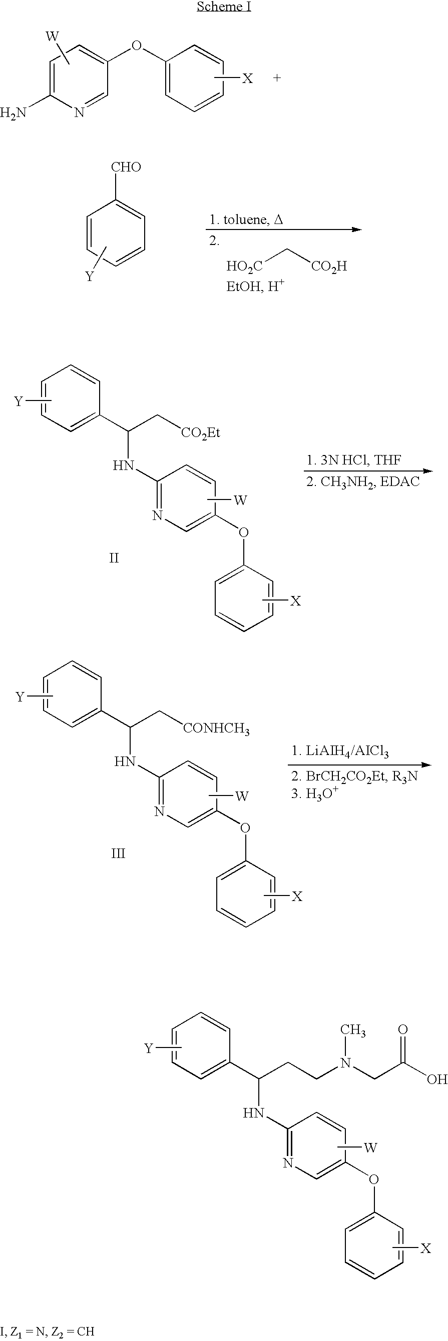

Scheme I illustrates methods of preparing compounds of the formula I wherein Z₁ is N and Z₂ is CH, and where R₁═CH₃. Methods analogous to these can be used to prepare compounds of the formula I wherein Z₂ is also N, or where R₁=hydrogen or C₁-C₆ alkyl. Such methods will be understood by those of skill in the art.

Referring to Scheme I, a compound of formula II is prepared by a procedure analogous to that found in Journal of the American Chemical Society, vol. 58, page 299, 1936. The appropriate aryloxy-substituted 2-aminopyridine, prepared by methods familiar to those skilled in the art, and a substituted benzaldehyde are combined in dry toluene and the reaction heated to drive off water. After cooling and evaporative removal of toluene, malonic acid and ethanol are added, and the reaction refluxed until product formation is maximal, generally 8 to 24 hours. The reaction is cooled and the product isolated by chromatography.

The compound of formula III is prepared from II by first hydrolyzing the ester group with acid, preferably a mineral acid such as hydrochloric acid, in a water miscible solvent such as tetrahydrofuran, dioxane, or ethanol. The acid formed is then converted to the N-methyl amide by standard coupling methods such as EDAC and HOBt in acetonitrile with a tertiary amine base as catalyst.

Conversion of III to the compound of formula I is accomplished by first reducing III using borane or alane (prepared from LiAlH4 and AlCl3) in an ethereal solvent such as tetrahydrofuran or dioxane at −70° C. to reflux for 1 to 24 hours. The resulting amine is then alkylated with ethyl bromoacetate in a polar solvent such as acetonitrile or dimethylformamide using a tertiary amine base as catalyst at room temperature to 70° C. for 1 to 48 hours, and then hydrolyzed with mineral acid, such as hydrochloric acid, in an ethereal solvent such as tetrahydrofuran or dioxane at a temperature from room temperature to reflux for 1 to 48 hours.

The compounds of formula I and the intermediates shown in the above reaction schemes can be isolated and purified by conventional procedures, such as recrystallization or chromatographic separation.

Compounds of formula I of this invention that contain basic substituents are capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained.

The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, ptoluenesulfonate and pamoate (i.e., 1,1′-methylene-bis-(2-hydroxy-3-naphthoate))salts.

All compounds of the invention have an acidic group and are capable of forming base salts with various pharmaceutically acceptable cations. Examples of such salts include the alkali metal or alkaline-earth metal salts and, particularly, the sodium and potassium salts. These salts are all prepared by conventional techniques.

The chemical bases which are used as reagents to prepare the pharmaceutically acceptable base salts of this invention are those which form non-toxic base salts with the herein described acidic derivatives. These particular non-toxic base salts include those derived form such pharmaceutically acceptable cations as sodium, potassium, calcium and magnesium, etc. These salts can easily be prepared by treating the aforementioned acidic compounds with an aqueous solution containing the desired pharmaceutically acceptable cation, and then evaporating the resulting solution to dryness, preferably under reduced pressure. Alternatively, they may also be prepared by mixing lower alkanoic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum production of yields of the desired final product.

The compounds of the present invention exhibit significant glycine transport inhibiting activity and therefore are of value in the treatment of a wide variety of clinical conditions that are characterized by the deficit of glutamergic neurotransmission in mammalian subjects, especially humans. Such conditions include the positive and negative symptoms of schizophrenia and other psychoses, and cognitive deficits.

The compounds of formula I of this invention can be administered via the oral, parenteral (such as subcutaneous, intravenous, intramuscular, intrasternal and infusion techniques), rectal, intranasal or topical routes to mammals. In general, these compounds are most desirably administered to humans in doses ranging from about 1 mg to about 2000 mg per day, although variations will necessarily occur depending upon the weight and condition of the subject being treated and the particular route of administration chosen. However, a dosage level that is in the range of from about 0.1 mg to about 20 mg per kg of body weight per day is most desirably employed. Nevertheless, variations may still occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects provided that such higher dose levels are first divided into several small doses for administration throughout the day.

The compounds of the present invention may be administered alone or in combination with pharmaceutically acceptable carriers or diluents by either of the above routes previously indicated, and such administration can be carried out in single or multiple doses. More particularly, the novel therapeutic agents of the invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various nontoxic organic solvents, etc. Moreover, oral pharmaceutical compositions can be suitably sweetened and/or flavored. In general, the therapeutically effective compounds of this invention are present in such dosage forms at concentration levels ranging about 5.0% to about 70% by weight.

For oral administration, tablets containing various excipients such as microcrystalline cellulose, sodium citrate, calcium carbonate, dicalcium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatine capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH>8) if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well-known to those skilled in the art. Additionally, it is also possible to administer the compounds of the present invention topically when treating inflammatory conditions of the skin and this may preferably be done by way of creams, jellies, gels, pastes, ointments and the like, in accordance with standard pharmaceutical practice.

The compounds of the present invention are assayed for their activity in inhibiting glycine reuptake in synaptosomes by first preparing synaptosomes and then measuring neurotransmitter reuptake activity as follows:

Male Sprague Dawley rats are decapitated and the brains removed. The whole brains are dissected out and placed in ice cold sucrose buffer; 1 gram in 20 mls (320 mM sucrose containing 1 mg/ml glucose, 0.1 mM EDTA and brought up to pH 7.4 with Tris base). The tissue is homogenized in a glass homogenizing tube with a Teflon™ pestle at 350 RPMS using a Potters homogenizer. The homogenate is centrifuged at 1000×g for 10 min at 4° C. The resulting supernatant is recentrifuged at 17,000×g for 20 min at 4° C. The final pellet is resuspended in an appropriate volume of sucrose buffer containing 5 mM alanine, to yield less than 10% uptake.

The uptake assays are conducted in 96 well matrix plates. Each well contains 25 μL of solvent, inhibitor or 10 mM glycine for nonspecific uptake, 200 μL of [³H]-glycine (40 nM final), made up in modified Krebs containing 5 mM alanine and glucose (1 mg/ml) and 25 μL of synaptosomes. The plates are then incubated at room temperature for the 15 min. The incubation is terminated by filtration through GF/B filters, using a 96 well Brandel Cell Harvester. The filters are washed with modified Krebs buffer and counted in a liquid scintillation counter or in a LKB Beta Plate counter. Compounds of the invention analyzed by this assay have been found to have significant activity in inhibiting glycine reuptake in synaptosomes, having IC₅₀ values of no greater than 50 nM.

The present invention is illustrated by the following examples. However, it should be understood that the invention is not limited to the specific details of these examples. Melting points were taken with a Buchi micro melting point apparatus and uncorrected. Infrared Ray absorption spectra (IR) were measured by a Shimadzu infrared spectrometer (IR-470). ¹H and ¹³C nuclear magnetic resonance spectra (NMR) were measured in CDCl₃ by a JEOL NMR spectrometer (JNM-GX270, 270 MHz for ¹H, 67.5 MHz for ¹³C) unless otherwise indicated and peak positions are expressed in parts per million (ppm) downfield from tetramethylsilane. The peak shapes are denoted as follows: s, singlet; d, doublet; t, triplet; m, multiplet; br, broad.

### EXAMPLE 1

### {Methyl-[3-phenyl-3-(5-p-tolyloxy-pyridin-2-ylamino)-propyl]-amino}-acetic acid

Prepared as shown in Scheme 1 in 75% yield as a white solid.

¹³C-NMR (δ, CDCl₃): 20.82, 32.64, 41.91, 56.96, 55.03, 57.71, 110.39, 117.98, 126.48, 128.1, 129.28, 130.61, 133.46, 133.80, 141.55, 145.74, 153,14, 155.01, 166.32, 169.86. MS (%): 406 (parent+1, 100). HRMS Calc'd. for C₂₄H₂₈N₃O₃: 406.2130. Found: 406.2134.

### EXAMPLE 2

### ({3-[5-(4-Methoxy-phenoxy)-pyridin-2-ylamino]-3-phenyl-propyl}-methyl-amino)-acetic acid

Prepared as in Scheme 1 in 21% yield as a white solid.

¹³C-NMR (δ, CDCl₃): 32.49, 41.77, 53.86, 55.12, 55.87, 57.65, 110.60, 115.27, 119.84, 126.32, 128.20, 129.32, 133.95, 141.32, 146.64, 150.16, 152.60, 156.34, 166.57, 169.97. MS (%): 422 (parent+1, 100). HRMS Calc'd. for C₂₄H₂₇N₃O₄: 422.2080. Found: 422.2083.

### EXAMPLE 3

### ({3-(4-Fluoro-phenyl)-3-[5-(4-methoxy-phenoxy)-pyridin-2-ylamino]-propyl}-methyl-amino)-acetic acid

Prepared as in Scheme 1 in 22.5% yield as a white solid.

¹³C-NMR (δ, CDCl₃): 32.70, 41.88, 53.92, 54.36, 55.87, 57.73, 110.34, 115.24, 116.04, 116.25, 119.73, 128.01, 131.70, 133.26, 137.43, 146.74, 150.37, 152.80, 161.18, 166.53, 169.91. MS (%): 440 (parent+1, 100). HRMS Calc'd. for C₂₄H₂₆FN₃O₄: 440.1986. Found: 440.2003.

### EXAMPLE 4

### {[3-(4-Fluoro-phenyl)-3-(5-p-tolyloxy-pyridin-2-ylamino)-propyl]-methyl-amino}-acetic acid

Prepared as in Scheme 1 in 39% yield as a white solid.

¹³C-NMR (δ, CDCl₃): 20.81, 32.59, 41.80, 53.99, 54.15, 57.84, 110.25, 115.98, 116.19, 117.91, 128.06, 128.14, 130.60, 133.29, 133.38, 133.47, 137.60, 145.80, 153.23, 155.10, 162.35 (J=245), 166.62, 169.82. MS (%): 424 (parent+1, 100). HRMS Calc'd. for C₂₄H₂₆FN₃O₃: 424.2036. Found: 424.2049.

### EXAMPLE 5

### {[3-(4-Methoxy-phenyl)-3-(5-p-tolyloxy-pyridin-2-ylamino)-propyl]-methyl-amino}-acetic acid

Prepared as in Scheme 1 in 79% yield as a white solid.

¹³C-NMR (δ, CDCl₃): 20.79, 32.55, 41.71, 53.96, 54.21, 55.43, 57.82, 110.25, 114.54, 117.78, 127.58, 130.54, 133.17, 133.62, 133.74, 145.48, 153.50, 155.28, 159.27, 166.72, 169.76. MS (%): 436 (parent+1, 100).

### EXAMPLE 6

### ({3-[5-(4-Chloro-phenoxy)-pyridin-2-ylamino]-3-phenyl-propyl}methyl-amino)-acetic acid

Prepared as in Scheme 1 in 62% yield as a white solid.

¹³C-NMR (δ, CDCl₃): 32.46, 41.85, 54.20, 54.68, 58.06, 110.18, 118.77, 126.39, 128.09, 128.41, 129.24, 130.00, 133.33, 135.04, 141.74, 144.57, 154.08, 156.48, 166.35, 169.70. MS (%): 426 (parent+1, 100). HRMS Calc'd. for C₂₃H₂₄ClN₃O₃: 426.1584. Found: 426.1601.

### EXAMPLE 7

### {[3-[5-(4-Chloro-phenoxy)-pyridin-2-ylamino]-3-(4-fluoro-phenyl)-propyl]-methyl-amino}-acetic acid

Prepared as in Scheme 1 in 15% yield as a white solid.

¹³C-NMR (δ, CDCl₃): 32.58, 41.97, 54.17, 57.91, (one carbon signal is missing or accidentally equivalent), 110.47, 116.06, 116.27, 118.94, 128.05, 128.14, 130.09, 133.84, 137.38, 144.82, 153.63, 156.20, 162.43 (d, J=246), 165.94, 169.73. MS (%): 444 (parent+1, 100).

### EXAMPLE 8

### ({3-[5-(3-Methoxy-phenoxy)-pyridin-2-ylamino]-3-phenyl-propyl}-methyl-amino)-acetic acid

Prepared as in Scheme 1 in 36% yield as a white solid.

¹³C-NMR (δ, CDCl₃): 32.53, 41.86, 54.36, 54.46, 55.54, 58.33, 103.65, 108.60, 109.42, 126.45, 127.86, 129.12, 130.39,1 32.47, 136.88, 142.28, 144.58, 154.46, 159.42, 161.11, 166.77, 169.54. MS (%): 422 (parent+1, 100). HRMS Calc'd. for C₂₄H₂₇N₃O₄: 422.2080. Found: 422.2083.

### EXAMPLE 9

### ({3-(4-Fluoro-phenyl)-3-[5-(3-methoxy-phenoxy)-pyridin-2-ylamino]-propyl}-methyl-amino)-acetic acid

Prepared as in Scheme 1 in 47% yield as a white solid.

¹³C-NMR (δ, CDCl₃): 32.59, 41.84, 53.64, 54.35, 55.53, 58.46, 103.64, 108.46, 109.37, 115.90 (d, J=20), 128.18 (d, J=4), 128.22, 128.54, 130.39, 132.04, 137.71, 138.39, 144.66, 154.57, 159.55, 160.98, 162.27 (d, J=245), 166.70, 169.39. MS (%): 440 (parent+1, 100). HRMS Calc'd. for C₂₄H₂₆FN₃O₄: 440.1986. Found: 440.1965.

### EXAMPLE 10

### ({3-[5-(4-Fluoro-phenoxy)-pyridin-2-ylamino]-3-phenyl-propyl}-methyl-amino)-acetic acid

Prepared as in Scheme 1 in 36.5% yield as a white solid.

¹³C-NMR (δ, CDCl₃): 32.60, 41.80, 54.24, 54.40, 58.49, 109.51, 116.38 (d, J=24), 118.70 (d, J=8), 126.48, 127.75, 128.54, 129.06, 131.25, 137.58, 142.63, 145.17, 154.20, 154.72, 158.53 (d, J=240), 166.66, 169.31. MS (%): 410 (parent+1, 100). HRMS Calc'd. for C₂₃H₂₄FN₃O₃: 410.1880. Found: 410.1887.

### EXAMPLE 11

### {[3-[5-(4-Fluoro-phenoxy)-pyridin-2-ylamino]-3-(4-fluoro-phenyl)-propyl]-methyl-amino}-acetic acid

Prepared as in Scheme 1 in 33% yield as a white solid.

¹³C-NMR (δ, CDCl₃): 32.51, 41.82, 53.73, 54.29, 58.29, 109.83, 115.93 (d, J=22), 116.49 (d, J=24), 118.89, 118.97, 128.11, 128.19, 128.53, 131.90, 136.23, 138.14, 145.44, 153.85, 154.18, 157.49, 159.89, 162.22 (d, J=245), 166.57, 169.49. MS (%): 428 (parent+1, 100). HRMS Calc'd. for C₂₃H₂₃F₂N₃O₃: 428.1786. Found: 428.1786.

### EXAMPLE 12

### ({3-[5-(3-Fluoro-phenoxy)-pyridin-2-ylamino]-3-phenyl-propyl}-methyl-amino)-acetic acid

Prepared as in Scheme 1 in 74% yield as a white solid.

¹³C-NMR (δ, CDCl₃): 32.32, 42.0, 54.44, 55.0, (one carbon signal missing or accidentally equivalent), 104.88 (d, J=25), 109.91 (d, J=20), 112.70, 126.48, 127.93, 129.15, 130.77, 130.87, 132.97, 136.65, 142.06, 143.93, 154.60, 159.52 (d, J=12), 163.62 (d, J=245). MS (%): 410 (parent+1, 100). HRMS Calc'd. for C₂₃H₂₄FN₃O₃: 410.1880. Found: 410.1886.

### EXAMPLE 13

### {[3-[5-(3-Fluoro-phenoxy)-pyridin-2-ylamino]-3-(4-fluoro-phenyl)-propyl]-methyl-amino}-acetic acid

Prepared as in Scheme 1 in 74% yield as a white solid.

¹³C-NMR (δ, CDCl₃): 32.56, 41.89, 53.78, 54.23, (one carbon signal missing or accidentally equivalent), 104.91 (d, J=25), 109.99 (d, J=21), 112.76, 112.74, 115.91, 116.11, 128.11, 128.18, 130.81, 130.90, 133.02, 136.59, 137.88, 144.15, 154.48, 159.47 (d, J=9), 162.29 (d, J=245), 163.62 (d, J=245). MS (%): 428 (parent+1, 100). HRMS Calc'd. for C₂₃H₂₃F₂N₃O₃: 428.1786. Found: 428.1786.

### EXAMPLE 14

### ({3-[5-(4-Methoxy-phenoxy)-6-methyl-pyridin-2-ylamino]-3-phenyl-propyl}-methyl-amino)-acetic acid

Prepared as in Scheme 1 in 61% yield as a white solid.

¹³C-NMR (δ, CDCl₃): 15.11, 32.55, 41.56, 53.59, 55.54, 55.87, 107.43, 115.17, 118.04, 126.35, 128.14, 129.28, 135.22, 141.44, 142.29, 143.51, 151.07, 152.51, 155.71. MS (%): 436 (parent+1, 100). HRMS Calc'd. for C₂₅H₂₉N₃O₄: 436.2236. Found: 436.2221.

### EXAMPLE 15

### ({3-(4-Fluoro-phenyl)-3-[5-(4-methoxy-phenoxy)-6-methyl-pyridin-2-ylamino]-propyl}-methyl-amino)-acetic acid

Prepared as in Scheme 1 in 80% yield as a white solid.

¹³C-NMR (δ, CDCl₃): 14.99, 32.67, 41.62, 53.56, 54.92, 55.89, 107.42, 115.21, 116.19 (d, J=21), 118.15, 128.05 (d, J=8), 135.33, 137.20, 142.51, 143.38, 150.92, 152.28, 155.82, 162.45 (d, J=245). MS (%): 454 (parent+1, 100). HRMS Calc'd. for C₂₅H₂₈FN₃O₄: 454.2142. Found: 454.2134.

### EXAMPLE 16

### ({3-[5-(4-Methoxy-phenoxy)-4-methyl-pyridin-2-ylamino]-3-phenyl-propyl}-methyl-amino)-acetic acid

Prepared as in Scheme 1 in 87% yield as a white solid.

¹³C-NMR (δ, CDCl₃): 17.42, 31.93, 42.09, 53.61, 54.41, 55.86, 115.06, 119.28, 120.22, 126.63, 128.12, 129.23, 131.19, 132.95, 142.21, 146.61, 151.36, 152.19, 155.74. MS (%): 436 (parent+1,1 00). HRMS Calc'd. for C₂₅H₂₉N₃O₄: 436.2236. Found: 436.2240.

### EXAMPLE 17

### ({3-(4-Fluoro-phenyl)-3-[5-(4-methoxy-phenoxy)-4-methyl-pyridin-2-ylamino]-propyl}-methyl-amino)-acetic acid

Prepared as in Scheme 1 in 57% yield as a white solid.

¹³C-NMR (δ, CDCl₃): 17.5, 31.87, 42.15, 52.0, 54.58, 55.86, 115.07, 115.95 (d, J=21), 119.31, 120.28, 128.41 (d, J=9), 131.16, 132.76, 138.22, 146.67, 151.30, 151.92, 155.78, 162.36, (J=245). MS (%): 454 (parent+1, 100). HRMS Calc'd. for C₂₅H₂₈FN₃O₄: 454.2146. Found: 454.2157.

1. A compound of the formula: 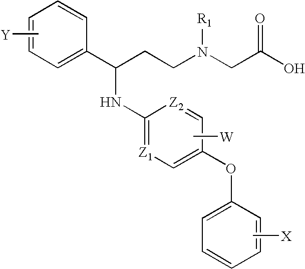
wherein Z₁ and Z₂ are independently carbon or nitrogen, provided that Z₂ is not carbon when Z₁ is carbon;
W, X and Y are each, independently, hydrogen, (C₁-C₆) alkyl optionally substituted with from one to seven fluorine atoms, (C₁-C₆)alkoxy optionally substituted with from one to seven fluorine atoms, with the proviso that the number of fluorine substituents on each of the (C₁-C₆) alkyl and (C₁-C₆) alkoxy groups does not exceed the number of positions in each of the (C₁-C₆) alkyl and (C₁-C₆) alkoxy groups that are available for substitution; carboxy; carbo-(C₁-C₆)alkoxy; carboxamido; (C₁-C₆)alkyl-thio; sulfoxyl; sulfonyl; halo; nitro; cyano; amino; (C₁-C₆) alkylamino and di[(C₁-C₆) alkyl]amino; and
R₁ is hydrogen or C₁-C₆ alkyl;
or a pharmaceutically acceptable salt thereof:

2. A compound according to claim 1, wherein Z₁ is N and Z₂ is CH.

3. A compound according to claim 1, wherein W, X and Y are each, independently, hydrogen, (C₁-C₆) alkyl optionally substituted with from one to seven fluorine atoms, (C₁-C₆)alkoxy optionally substituted with from one to seven fluorine atoms; with the proviso that the number of fluorine substituents on each of the (C₁-C₆) alkyl and (C₁-C₆) alkoxy groups does not exceed the number of positions in each of the (C₁-C₆) alkyl and (C₁-C₆) alkoxy groups that are available for substitution; alkylsulfonyl; halo; (C₁-C₆) alkylamino and di[(C₁-C₆) alkyl]amino.

4. A compound according to claim 1, wherein Y is selected from fluoro, bromo, chloro, methyl, ethyl, methoxy, ethoxy, phenyl, benzyl, and acetyl.

5. A compound according to claim 1, wherein X is 4-trifluoromethyl, 4-methyl, 4-methoxy, 4-phenyl, 4-benzyl, 3-methyl, 3-fluoro, 3-methoxy, 3-chloro or 4-chloro.

6. A compound according to claim 1, wherein W is selected from trifluoromethyl, methyl, methoxy, phenyl, benzyl, or chloro.

7. A compound according to claim 1, wherein R₁ is methyl.

8. A compound selected from the group consisting of:
{Methyl-[3-phenyl-3-(5-p-tolyloxy-pyridin-2-ylamino)-propyl]-amino}-acetic acid;
({3-[5-(4-Methoxy-phenoxy)-pyridin-2-ylamino]-3-phenyl-propyl}-methyl-amino)-acetic acid;
({3-(4-Fluoro-phenyl)-3-[5-(4-methoxy-phenoxy)-pyridin-2-ylamino]-propyl}-methyl-amino)-acetic acid;
{[3-(4-Fluoro-phenyl)-3-(5-p-tolyloxy-pyridin-2-ylamino)-propyl]-methyl-amino}-acetic acid;
{[3-(4-Methoxy-phenyl)-3-(5-p-tolyloxy-pyridin-2-ylamino)-propyl]-methyl-amino}-acetic acid;
({3-[5-(4-Chloro-phenoxy)-pyridin-2-ylamino]-3-phenyl-propyl}-methyl-amino)-acetic acid;
{[3-[5-(4-Chloro-phenoxy)-pyridin-2-ylamino]-3-(4-fluoro-phenyl)-propyl]-methyl-amino}-acetic acid;
({3-[5-(3-Methoxy-phenoxy)-pyridin-2-ylamino]-3-phenyl-propyl}-methyl-amino)-acetic acid;
({3-(4-Fluoro-phenyl)-3-[5-(3-methoxy-phenoxy)-pyridin-2-ylamino]-propyl}-methyl-amino)-acetic acid;
({3-[5-(4-Fluoro-phenoxy)-pyridin-2-ylamino]-3-phenyl-propyl}-methyl-amino)-acetic acid;
{[3-[5-(4-Fluoro-phenoxy)-pyridin-2-ylamino]-3-(4-fluoro-phenyl)-propyl]-methyl-amino}-acetic acid;
({3-[5-(3-Fluoro-phenoxy)-pyridin-2-ylamino]-3-phenyl-propyl}-methyl-amino)-acetic acid;
{[3-[5-(3-Fluoro-phenoxy)-pyridin-2-ylamino]-3-(4-fluoro-phenyl)-propyl]-methyl-amino}-acetic acid;
({3-[5-(4-Methoxy-phenoxy)-6-methyl-pyridin-2-ylamino]-3-phenyl-propyl}-methyl-amino)-acetic acid;
({3-(4-Fluoro-phenyl)-3-[5-(4-methoxy-phenoxy)-6-methyl-pyridin-2-ylamino]-propyl}-methyl-amino)-acetic acid;
({3-[5-(4-Methoxy-phenoxy)-4-methyl-pyridin-2-ylamino]-3-phenyl-propyl}-methyl-amino)-acetic acid; and
({3-(4-Fluoro-phenyl)-3-[5-(4-methoxy-phenoxy)4-methyl-pyridin-2-ylamino]-propyl}-methyl-amino)-acetic acid;
{Methyl-[3-phenyl-3-(5-p-tolyloxy-pyrimidin-2-ylamino)-propyl]-amino}-acetic acid;
({3-[5-(4-Methoxy-phenoxy)-pyrimidin-2-ylamino]-3-phenyl-propyl}-methyl-amino)-acetic acid;
({3-(4-Fluoro-phenyl)-3-[5-(4-methoxy-phenoxy)-pyrimidin-2-ylamino]-propyl}-methyl-amino)-acetic acid;
{[3-(4-Fluoro-phenyl)-3-(5-p-tolyloxy-pyrimidin-2-ylamino)-propyl]-methyl-amino}-acetic acid;
{[3-(4-Methoxy-phenyl)-3-(5-p-tolyloxy-pyrimidin-2-ylamino)-propyl}-methyl-amino}-acetic acid; and
({3-[5-(4-Chloro-phenoxy)-pyrimidin-2-ylamino]-3-phenyl-propyl}-methyl-amino)-acetic acid.

9. A method of treating a disorder or condition selected from psychosis, schizophrenia, conduct disorder, disruptive behavior disorder, bipolar disorder, psychotic episodes of anxiety, anxiety associated with psychosis, psychotic mood disorders selected from severe major depressive disorder; mood disorders associated with psychotic disorders selected from acute mania, depression associated with bipolar disorder and mood disorders associated with schizophrenia; behavioral manifestations of mental retardation, conduct disorder and autistic disorder; movement disorders selected from Tourette's syndrome, akinetic-rigid syndrome, movement disorders associated with Parkinson's disease, tardive dyskinesia, and other drug-induced and neurodegeneration-based dyskinesias; neurodegenerative diseases; attention deficit hyperactivity disorder; cognitive disorders selected from dementias, age-related dementia and senile dementia of the Alzheimer's type; and memory disorders in a mammal, comprising administering to a mammal in need of such treatment an amount of a compound of according to claim 1 that is effective in treating such condition or disorder.

10. The method of claim 9, wherein the disorder or condition is selected from the group consisting of movement disorders selected from Tourette's syndrome, akinetic-rigid syndrome, movement disorders associated with Parkinson's disease, tardive dyskinesia, and multiple sclerosis.

11. A pharmaceutical composition for treating a disorder or condition selected from psychosis, schizophrenia, conduct disorder, disruptive behavior disorder, bipolar disorder, psychotic episodes of anxiety, anxiety associated with psychosis, psychotic mood disorders selected from severe major depressive disorder; mood disorders associated with psychotic disorders selected from acute mania and depression associated with bipolar disorder, and mood disorders associated with schizophrenia; behavioral manifestations of mental retardation, conduct disorder and autistic disorder; movement disorders selected from Tourette's syndrome, akinetic-rigid syndrome, movement disorders associated with Parkinson's disease, tardive dyskinesia and other drug-induced and neurodegeneration-based dyskinesias; neurodegenerative diseases; attention deficit hyperactivity disorder; cognitive disorders selected from dementias, age-related dementia and senile dementia of the Alzheimer's type; and memory disorders in a mammal, comprising an amount of a compound according to claim 1 that is effective in treating such disorder or condition.

12. The composition of claim 11, wherein the disorder or condition is selected from the group consisting of movement disorders selected from Tourette's syndrome, akinetic-rigid syndrome, movement disorders associated with Parkinson's disease, tardive dyskinesia, and multiple sclerosis.

13. A method of treating a disorder or condition selected from psychosis, schizophrenia, conduct disorder, disruptive behavior disorder, bipolar disorder, psychotic episodes of anxiety, anxiety associated with psychosis, psychotic mood disorders selected from severe major depressive disorder; mood disorders associated with psychotic disorders selected from acute mania and depression associated with bipolar disorder, and mood disorders associated with schizophrenia; behavioral manifestations of mental retardation, conduct disorder and autistic disorder; movement disorders selected from Tourette's syndrome, akinetic-rigid syndrome, and movement disorders associated with Parkinson's disease, tardive dyskinesia, and other drug-induced and neurodegeneration-based dyskinesias; neurodegenerative diseases; attention deficit hyperactivity disorder; cognitive disorders selected from dementias, age-related dementia, and senile dementia of the Alzheimer's type; and memory disorders in a mammal, including a human, comprising administering to a mammal in need of such treatment a glycine transport inhibiting amount of a compound according to claim 1.

14. The method of claim 13, wherein the disorder or condition is selected from the group consisting of movement disorders selected from Tourette's syndrome, akinetic-rigid syndrome, movement disorders associated with Parkinson's disease, tardive dyskinesia, and multiple sclerosis.

15. A pharmaceutical composition for treating a disorder or condition selected from psychosis, schizophrenia, conduct disorder, disruptive behavior disorder, bipolar disorder, psychotic episodes of anxiety, anxiety associated with psychosis, psychotic mood disorders selected from severe major depressive disorder; mood disorders associated with psychotic disorders selected from acute mania and depression associated with bipolar disorder, and mood disorders associated with schizophrenia; behavioral manifestations of mental retardation, conduct disorder and autistic disorder; movement disorders selected from Tourette's syndrome, akinetic-rigid syndrome, movement disorders associated with Parkinson's disease, tardive dyskinesia and other drug-induced and neurodegeneration-based dyskinesias; neurodegenerative diseases; attention deficit hyperactivity disorder; cognitive disorders selected from dementias, age-related dementia and senile dementia of the Alzheimer's type; and memory disorders in a mammal, including a human, comprising a glycine transport-inhibiting amount of a compound according to claim 1.

16. The composition of claim 15, wherein the disorder or condition is selected from the group consisting of movement disorders selected from Tourette's syndrome, akinetic-rigid syndrome, movement disorders associated with Parkinson's disease, tardive dyskinesia, and multiple sclerosis.
